(19) United States
(12) Patent Application Publication
(10) Pub. No.: US 2017/0196824 A1
(43) Pub. Date: Jul. 13, 2017

(54) COMPOSITIONS AND METHODS OF USE OF BETA-HYDROXY-BETA-METHYLBUTYRATE (HMB) FOR JOINT STABILITY

(71) Applicant: Metabolic Technologies, Inc., Ames, IA (US)

(72) Inventors: John Fuller, JR., Zearing, IA (US); John Rathmacher, Story City, IA (US); Emily Harris, Des Moines, IA (US); Shawn Baier, Polk City, IA (US)

(21) Appl. No.: 15/405,880

(22) Filed: Jan. 13, 2017

Related U.S. Application Data
(60) Provisional application No. 62/278,252, filed on Jan. 13, 2016.

(51) Int. Cl. A61K 31/19

(52) U.S. Cl. CPC A61K 31/19

(57) ABSTRACT

The present invention provides a composition comprising HMB. Methods of administering HMB to an animal are also described. HMB is administered to decrease joint instability, increase joint stability, decrease joint stiffness, improve joint function, improve joint health, improve balanced movement and improve skeletal joint stability during muscle movement. HMB is administered to increase muscle strength and/or muscle mass in a side of the body contralateral to joint inflammation, joint damage, and/or joint injury.

This application claims priority to United States Provisional Patent Application No. 62/278,252 filed Jan. 13, 2016 and herein incorporates the provisional application by reference.

### BACKGROUND OF THE INVENTION

1. Field

The present invention relates to a composition comprising β-hydroxy-62 -methylbutyrate (HMB) and methods of using HMB to reduce joint instability, increase joint stability, and improve balanced movement.

2. Background

As animals age, their body condition changes and the musculoskeletal system that surrounds the joints to provide joint stability can weaken. Joint instability can lead to a decrease in range of motion and flexibility and an increase in inflammation and pain. The result is a decrease in activity and inability to perform daily tasks such as climbing stairs.

Resistance to joint instability is accomplished through healthy tendons, ligaments, fascia and muscle tissues which surround the joint, and can be important determinants in maintaining a pain-free range of motion and flexibility.

The neuromuscular system consists of neuromuscular junctions and is responsible for a coordinated and forceful muscle contraction. Together these systems provide for balanced movement and stability of the skeletal joints during muscle movements. Targeted nutritional therapies may be able to help maintain this balance and thus mobility and quality of life in older animals, including but not limited to humans and companion animals. The nutritional regimen comprising administration of HMB as described below improves balanced movement, decreases joint stiffness, assists in maintaining health joints, and improved joint stability resulting in increased activity and improved quality of life.

HMB

Alpha-ketoisocaproate (KIC) is the first major and active metabolite of leucine. A minor product of KIC metabolism is β-hydroxy-β-methylbutyrate (HMB). HMB has been found to be useful within the context of a variety of applications. Specifically, in U.S. Pat. No. 5,360,613 (Nissen), HMB is described as useful for reducing blood levels of total cholesterol and low-density lipoprotein cholesterol. In U.S. Pat. No. 5,348,979 (Nissen et al.), HMB is described as useful for promoting nitrogen retention in humans. U.S. Pat. No. 5,028,440 (Nissen) discusses the usefulness of HMB to increase lean tissue development in animals. Also, in U.S. Pat. No. 4,992,470 (Nissen), HMB is described as effective in enhancing the immune response of mammals. U.S. Pat. No. 6,031,000 (Nissen et al.) describes use of HMB and at least one amino acid to treat disease-associated wasting.

The use of HMB to suppress proteolysis originates from the observations that leucine has protein-sparing characteristics. The essential amino acid leucine can either be used for protein synthesis or transaminated to the α-ketoacid (α-ketoisocaproate, KIC). In one pathway, KIC can be oxidized to HMB and this account for approximately 5% of leucine oxidation. HMB is superior to leucine in enhancing muscle mass and strength. The optimal effects of HMB can be achieved at 3.0 grams per day when given as calcium salt of HMB, or 0.038 g/kg of body weight per day, while those of leucine require over 30.0 grams per day.

Once produced or ingested, HMB appears to have two fates. The first fate is simple excretion in urine. After HMB is fed, urine concentrations increase, resulting in an approximate 20-50% loss of HMB to urine. Another fate relates to the activation of HMB to HMB-CoA. Once converted to HMB-CoA, further metabolism may occur, either dehydration of HMB-CoA to MC-CoA, or a direct conversion of HMB-CoA to HMG-CoA, which provides substrates for intracellular cholesterol synthesis. Several studies have shown that HMB is incorporated into the cholesterol synthetic pathway and could be a source for new cell membranes that are used for the regeneration of damaged cell membranes. Human studies have shown that muscle damage following intense exercise, measured by elevated plasma CPK (creatine phosphokinase), is reduced with HMB supplementation within the first 48 hrs. The protective effect of HMB lasts up to three weeks with continued daily use. Numerous studies have shown an effective dose of HMB to be 3.0 grams per day as CaHMB (calcium HMB) (˜38 mg/kg body weight-day⁻¹). This dosage increases muscle mass and strength gains associated with resistance training, while minimizing muscle damage associated with strenuous exercise. HMB has been tested for safety, showing no side effects in healthy young or old adults. HMB in combination with L-arginine and L-glutamine has also been shown to be safe when supplemented to AIDS and cancer patients.

Recently, HMB free acid, a new delivery form of HMB, has been developed. This new delivery form has been shown to be absorbed quicker and have greater tissue clearance than CaHMB. The new delivery form is described in U.S. Patent Publication Serial No. 20120053240 which is herein incorporated by reference in its entirety.

Current evidence suggests that HMB acts by speeding regenerative capacity of skeletal muscle following high intensity or prolonged exercise. When training and/or diet are controlled, HMB can lower indices of skeletal muscle damage and protein breakdown in a dose-dependent fashion. Recently, HMB in a free acid form (HMB-FA) has been developed with improved bioavailability. Initial studies have shown that this form of HMB supplementation results in approximately double the plasma levels of HMB in about one-quarter the time after administration when compared with the presently available form, calcium HMB. Further, HMB-FA given 30 minutes prior to an acute bout of high volume resistance training was able to attenuate indices of muscle damage and improve perceived recovery in resistance trained athletes (61). Moreover, acute ingestion of 2.4 grams of HMB-FA increases skeletal muscle protein synthesis and decreases protein breakdown by +70% and −56% respectively.

The effects of HMB on muscle are well documented. It is known that HMB supplementation leads to increased muscle mass and strength and can result in aerobic improvement. The present invention comprises a composition of HMB and methods of use of HMB to result in decreasing joint instability, increasing joint stability, improving balanced movement and improving skeletal joint stability during muscle movement.

Joint issues can have a significant impact on quality of life. If joints become unstable, the joint may eventually change shape and deformities can develop. Inflammation and pain are also results of unstable joints. Muscles, tendons, ligaments and cartilage work together to ensure smooth joint function, to guide and align joints through their range of motion and to make movement possible. Joint instability can have a variety of effects, including trouble getting up and down, slow or stiff movement, difficulty climbing stairs, limping and/or favoring one limb over the other.

Muscle mass and muscle contractions are essential to maintaining functional joints. The muscles counteract loading forces and maintain the functioning of the joint. Muscle recruitment as a joint stabilizer depends upon the force that the muscle can exert which is based upon many factors; however, muscle mass or muscle size is considered to be one of the more important determining factors in maintaining joint stability and proper functioning. The loss of muscle mass can lead to joint instability and affect the joint function which may then lead to inflammation and eventually arthritis.

If a joint or joints on one side of the body are affected by joint instability, inflammation, or injury, maintaining or improving contralateral strength helps overcome impaired movement resulting from the aforementioned joint instability, inflammation or injury and in particular helps compensate for the lack of balance due to the impaired muscle and joint of the ipsilateral side (Jeon K. Comparison of knee laxity and isokinetic muscle strength in patients with a posterior cruciate ligament injury. J Phys Ther Sci 2016; 28:831-836).

Muscle strengthening in patients with knee instability and low muscle strength has been shown to be important before undergoing knee stabilization training. (Knoop J, van der L M, Roorda L D et al. Knee joint stabilization therapy in patients with osteoarthritis of the knee and knee instability: subgroup analyses in a randomized, controlled trial. J Rehabil Med 2014; 46(7):703-707). Improved leg strength is beneficial in patients with joint atrophy and helps them conduct daily activities such as walking and climbing stairs more readily (Knoop J, Steultjens M P, Roorda L D et al. Improvement in upper leg muscle strength underlies beneficial effects of exercise therapy in knee osteoarthritis: secondary analysis from a randomised controlled trial. Physiotherapy 2015; 101(2):171-177).

Strength is important for balanced movement. It has been shown that declines in lower extremity strength were associated with a decline in balance. Older adults with greater strength had less decline in balance (Messier S P, Glasser J L, Ettinger W H, Jr., Craven T E, Miller M E. Declines in strength and balance in older adults with chronic knee pain: a 30-month longitudinal, observational study. Arthritis Rheum 2002; 47(2):141-148).

Joint issues include joint inflammation, joint damage, joint injury, and degenerative joint issues such as osteoarthritis and hip and/or elbow dysplasia. Thus, the need exists for a composition and methods of use of the composition to address joint issues, stabilize joints, reduce joint stiffness and improve balanced movement.

### SUMMARY OF THE INVENTION

One object of the present invention is to provide a composition for use in decreasing joint instability.

A further object of the present invention is to provide a composition for use in increasing joint stability.

Another object of the present invention is to provide a composition to provide for balanced movement and stability of the skeletal joints during muscle movement.

Another object of the present invention is to provide methods of administering a composition for use in decreasing joint instability.

An additional object of the present invention is to provide methods of administering a composition for increasing joint stability.

A further object of the present invention is to provide methods of administering a composition for improved balanced movement and stability of the skeletal joints during muscle movement.

Another object of the present invention is to provide methods of administering a composition for reducing joint stiffness.

An additional object of the present invention is to provide methods of administering a composition for maintaining and/or improving contralateral strength wherein there is joint damage, joint inflammation, joint weakening, or joint injury to the ipsilateral side.

These and other objects of the present invention will become apparent to those skilled in the art upon reference to the following specification, drawings, and claims.

The present invention intends to overcome the difficulties encountered heretofore. To that end, a composition comprising HMB is provided. The composition is administered to a subject in need thereof. All methods comprise administering to the animal HMB. The subjects included in this invention include humans and non-human mammals, including companion animals such as dogs, cats and horses.

### BRIEF DESCRIPTION OF THE FIGURES

FIG. 1 is a graph depicting mean muscle mass.

FIG. 2 is a graph depicting mean muscle strength.

### DETAILED DESCRIPTION OF THE INVENTION

It has been surprisingly and unexpectedly discovered that HMB improves joint stability, lessens joint stiffness, preserves and/or improves healthy joint function, and improves balanced movement. The present invention comprises a composition of HMB and methods of use of HMB to result in decreases in joint instability, improvement in joint stability, lessening of joint stiffness, promotion of healthy joints, increased joint motion range, improvement in balanced movement and stability of skeletal joints.

This composition can be used on all age groups seeking these outcomes. This composition can also be used in humans and non-human mammals, including but not limited to companion animals such as dogs, cats and horses.

HMB

β-hydroxy-β-methylbutyric acid, or β-hydroxy-isovaleric acid, can be represented in its free acid form as (CH₃)₂(OH)CCH₂COOH. The term “HMB” refers to the compound having the foregoing chemical formula, in both its free acid and salt forms, and derivatives thereof. Derivatives include metabolites, esters and lactones. While any form of HMB can be used within the context of the present invention, preferably HMB is selected from the group comprising a free acid, a salt, an ester, and a lactone. HMB esters include methyl and ethyl esters. HMB lactones include isovalaryl lactone. HMB salts include sodium salt, potassium salt, chromium salt, calcium salt, magnesium salt, alkali metal salts, and earth metal salts.

Methods for producing HMB and its derivatives are well-known in the art. For example, HMB can be synthesized by oxidation of diacetone alcohol. One suitable procedure is described by Coffman et al., J. Am. Chem. Soc. 80:2882-2887 (1958). As described therein, HMB is synthesized by an alkaline sodium hypochlorite oxidation of diacetone alcohol. The product is recovered in free acid form, which can be converted to a salt. For example, HMB can be prepared as its calcium salt by a procedure similar to that of Coffman et al. (1958) in which the free acid of HMB is neutralized with calcium hydroxide and recovered by crystallization from an aqueous ethanol solution. The calcium salt of HMB is commercially available from Metabolic

Technologies, Ames, Iowa.

Calcium β-hydroxy-β-methylbutyrate (HMB) Supplementation

More than 2 decades ago, the calcium salt of HMB was developed as a nutritional supplement for humans. Numerous studies have shown that CaHMB supplementation improves muscle mass and strength gains in conjunction with resistance-exercise training, and attenuates loss of muscle mass in conditions such as cancer and AIDS. Nissen and Sharp performed a meta-analysis of supplements used in conjunction with resistance training and found that HMB was one of only two supplements that had clinical studies showing significant increases in strength and lean mass with resistance training. Studies have shown that 38 mg of CaHMB per kg of body weight appears to be an efficacious dosage for an average person.

In addition to strength and muscle mass gains, CaHMB supplementation also decreases indicators of muscle damage and protein degradation. Human studies have shown that muscle damage following intense exercise, measured by elevated plasma CPK (creatine phosphokinase), is reduced with HMB supplementation. The protective effect of HMB has been shown to manifest itself for at least three weeks with continued daily use. In vitro studies in isolated rat muscle show that HMB is a potent inhibitor of muscle proteolysis especially during periods of stress. These findings have been confirmed in humans; for example, HMB inhibits muscle proteolysis in subjects engaging in resistance training.

The molecular mechanisms by which HMB decreases protein breakdown and increases protein synthesis have been reported. Eley et al conducted in vitro studies which have shown that HMB stimulates protein synthesis through mTOR phosphorylation. Other studies have shown HMB decreases proteolysis through attenuation of the induction of the ubiquitin-proteasome proteolytic pathway when muscle protein catabolism is stimulated by proteolysis inducing factor (PIF), lipopolysaccharide (LPS), and angiotensin II. Still other studies have demonstrated that HMB also attenuates the activation of caspases-3 and -8 proteases. Taken together these studies indicate that HMB supplementation results in increased lean mass and the accompanying strength gains through a combination of decreased proteolysis and increased protein synthesis.

HMB Free Acid Form

In most instances, the HMB utilized in clinical studies and marketed as an ergogenic aid has been in the calcium salt form. Recent advances have allowed the HMB to be manufactured in a free acid form for use as a nutritional supplement. Recently, a new free acid form of HMB was developed, which was shown to be more rapidly absorbed than CaHMB, resulting in quicker and higher peak serum HMB levels and improved serum clearance to the tissues. HMB in the free acid form is represented by the name “HMB-acid.”

HMB free acid may therefore be a more efficacious method of administering HMB than the calcium salt form, particularly when administered directly preceding intense exercise. HMB free acid initiated 30 min prior to an acute bout of exercise was more efficacious in attenuating muscle damage and ameliorating inflammatory response than CaHMB. One of ordinary skill in the art, however, will recognize that this current invention encompasses HMB in any form.

HMB in any form may be incorporated into the delivery and/or administration form in a fashion so as to result in a typical dosage range of about 0.5 grams HMB to about 30 grams HMB in a 24 hour period. HMB can also be administered in a dosage range of 0.01 to 0.2 g of HMB per kilogram of body weight in a 24 hour period.

The HMB itself can be present in any form; for example, CaHMB is typically a powder that can be incorporated into any delivery form, while HMB-acid is typically a liquid or gel that can be incorporated into any delivery form. Non-limiting examples of delivery forms include pills, tablets, capsules, gelcaps, liquids, beverages, solids and gels. For animals such as companion animals, HMB can be incorporated into feed, including commercial pet food, chews and treats, alone with the previously listed delivery forms.

The term administering or administration includes providing a composition to a mammal, consuming the composition and combinations thereof.

When the composition is administered orally in an edible form, the composition is preferably in the form of a dietary supplement, foodstuff or pharmaceutical medium, more preferably in the form of a dietary supplement or foodstuff. Any suitable dietary supplement or foodstuff comprising the composition can be utilized within the context of the present invention. One of ordinary skill in the art will understand that the composition, regardless of the form (such as a dietary supplement, foodstuff or a pharmaceutical medium), may include amino acids, proteins, peptides, carbohydrates, fats, sugars, minerals and/or trace elements.

In order to prepare the composition as a dietary supplement or foodstuff, the composition will normally be combined or mixed in such a way that the composition is substantially uniformly distributed in the dietary supplement or foodstuff. Alternatively, the composition can be dissolved in a liquid, such as water.

The composition of the dietary supplement may be a powder, a gel, a liquid or may be tabulated or encapsulated.

Although any suitable pharmaceutical medium comprising the composition can be utilized within the context of the present invention, preferably, the composition is combined with a suitable pharmaceutical carrier, such as dextrose or sucrose.

Furthermore, the composition of the pharmaceutical medium can be intravenously administered in any suitable manner. For administration via intravenous infusion, the composition is preferably in a water-soluble non-toxic form. Intravenous administration is particularly suitable for hospitalized patients that are undergoing intravenous (IV) therapy. For example, the composition can be dissolved in an IV solution (e.g., a saline or glucose solution) being administered to the patient. Also, the composition can be added to nutritional IV solutions, which may include amino acids, peptides, proteins and/or lipids. The amounts of the composition to be administered intravenously can be similar to levels used in oral administration. Intravenous infusion may be more controlled and accurate than oral administration.

Methods of calculating the frequency by which the composition is administered are well-known in the art and any suitable frequency of administration can be used within the context of the present invention (e.g., one 6 g dose per day or two 3 g doses per day) and over any suitable time period (e.g., a single dose can be administered over a five minute time period or over a one hour time period, or, alternatively, multiple doses can be administered over an extended time period). HMB can be administered over an extended period of time, such as weeks, months or years. The composition can be administered in individual servings comprising one or more than one doses/individual servings per day, to make a daily serving comprising the total amount of the composition administered in a day or 24 hour period.

Any suitable dose of HMB can be used within the context of the present invention. Methods of calculating proper doses are well known in the art.

In general, an amount of HMB in the levels sufficient to result in decreased joint instability, increased joint stability, decreased joint stiffness, increased or maintained contralateral strength, improved balanced movement and stability of the skeletal joints during muscle movement is provided.

Case Study

The following Example will illustrate the invention in further detail. It will be readily understood that the composition of the present invention, as generally described and illustrated in the Example herein, could be synthesized in a variety of formulations and dosage forms, and applied across any age range and any species including humans and non-human animals. Thus, the following more detailed description of the presently preferred embodiments of the methods, formulations and compositions of the present invention are not intended to limit the scope of the invention, as claimed, but it is merely representative of the presently preferred embodiments of the invention.

An eleven-year old female golden retriever weighing approximately 70 pounds was diagnosed with arthritis in the joints and was experiencing apparent paid and significantly decreased activity. Prior to developing arthritis, the dog climbed stairs, jumped in and out of a vehicle, and climbed up on furniture.

CaHMB in a capsule form was administered to the dog in the amount of 2 grams/day, in 1 gram doses with normal diet.

Soon after the treatment regimen began, the dog began climbing stairs, running limited distances and climbing onto furniture.

Approximately two months after beginning the HMB regimen, the dog developed seizure activity related to a growing brain tumor. Treatment for the seizure activity included phenobarbital, gabapentin, and potassium bromide, which caused severe ataxia such that the dog could no longer stand up from a lying down position without assistance, climb stairs, or get onto furniture. HMB administration was temporarily suspended for approximately two weeks upon the onset of seizures and the introduction of the anti-seizure medication. Upon resuming the HMB regimen, the dog began standing up without assistance, and was able to climb stairs without significant assistance. The owner observed significant improvement in the ataxia as well.

Administration of HMB to this dog resulted in increased joint stability, decreased joint instability, improved balanced movement and stability of skeletal joints allowing her to resume daily activities such as climbing stairs and standing without assistance. Her owners perceived her quality of life as being significantly improved.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

### Experimental Examples

Twenty-one female New Zealand White rabbits (43 weeks old) (Covance Research Products, Inc., Greenfield, Ind.) were used in the 8-week study conducted at the University of Calgary (Calgary, Alberta, Canada). Botox was used as an agent to replicate soft tissue trauma. The rabbits were randomly assigned to one of the following treatment groups:
(1) Control group: saline injection unilaterally (n=7);
(2) Botox group: single Botox injection unilaterally (n=7);
(3) Botox+HMB group: single Botox injection unilaterally+CaHMB food supplementation throughout the experimental period (n=7). HMB=Calcium salt of β-hydroxy-β-methylbutyrate

Group 1 rabbits served as controls and received intramuscular saline injection randomized (right or left) to the quadriceps musculature. The total volume of injected saline was the same as the total volume of Botox. Groups 2 and 3 rabbits received a one-time intramuscular Botox injection and data was collected eight weeks following the injection.

Saline and Botox Injections: The rabbits in the saline group were injected with 0.175 ml saline/kg body weight. The rabbits in the Botox and Boxtox+HMB groups were injected with a Clostridium botulinum type-A (BTX-A) neurotoxin complex (Botox®, Allergan, Inc. Toronto, Ontario, Canada). Briefly, the lyophilized toxin (100 U/vial) was reconstituted with 0.9% sodium chloride to a concentration of 20 U/ml. The right or left leg was chosen at random and 3.5 U/kg body weight was injected into the quadriceps muscle. The anterior compartment of the thigh was isolated by palpation and the quadriceps muscle visually divided into superior and inferior halves. Each half was then each subdivided into a medial, lateral, and central section. One sixth of the BTX-A dose was injected into each section to increase the diffusion and to equally distribute the BTX-A through the quadriceps musculature.

Diet: Group 1 and 2 rabbits received a high fiber diet (Laboratory Rabbit Diet HF 5326, LabDiet, Richmond, Ind.), while group 3 rabbits received the same base diet custom formulated with 0.44% CaHMB (Metabolic Technologies, Inc., Ames, Iowa, USA). Body weights and feed intakes were recorded weekly.

Quadriceps Muscle Strength: Knee extensor strength was assessed by stimulating the quadriceps via a femoral nerve cuff electrode implanted prior to testing. Following nerve cuff implantation, rabbits were secured in a stereotactic frame using bone pins at the pelvis and femoral condyles. Isometric knee extensor forces at 80°, 100°, and 120° of knee flexion were measured using a strain-gauged, calibrated bar placed over the distal portion of the rabbit's tibia.

Stimulation of the knee extensor musculature (Grass S8800 stimulator; Astro-Med Inc., Longueil, Quebec, Canada) was performed at a voltage three times higher than the alpha motor neuron threshold, to ensure activation of all motor units. Stimulation duration was 500 ms, pulse duration 0.1 ms, and the measurements were made at two frequencies of stimulation, 100 Hz and 200 Hz. A two minute rest period was given between stimulations to prevent muscle fatigue.

Quadriceps Muscle Mass: Following the 8-week feeding period, the rabbits were killed by an overdose of Euthanyl (MTC Pharmaceutical, Cambridge, Ontario, Canada) given into the heart. The wet muscle mass of the quadriceps muscle was determined immediately after the animals were sacrificed. The quadriceps muscle group was removed and the rectus femoris (RF), vastus medialis (VM), vastus lateralis (VL) and small vastus lateralis (SVL) were separated and individually weighed using a commercial scale with an accuracy of 0.01 g.

Statistical Analysis

The data were analyzed using Proc GLM in SAS (SAS for Windows 9.4, SAS Institute, Inc., Cary, N.C.). The model used the main effect of treatment. For actual muscle weights and strength measurements, body weight of the rabbit was used as a covariate. Means reported are Least Squares means and the standard error of the mean was calculated from the mean square of the error term of the main effect model. The p-values given for overall treatment are from the main effect model while individual means were compared using the Least Square Means predicted difference. A p-value≦0.05 indicates significance while 0.05<p<0.10 indicates a definite trend towards significance in the data.

Body Weights and Feed Intake

There were no differences in average body weights at the time the muscle measurements were made. The control, botox, and botox+HMB rabbits weighed 4.17±0.08, 4.04±0.13, and 3.99±0.10 kg, respectively. Feed intake over the 8-week study averaged 168.5±7.6, 135.5±7.9, and 129.4±8.0 g/day for the control, botox, and botox+HMB rabbits, respectively. The botox and botox+HMB groups ate significantly less feed due to decreased feed intake after the botox injections (p<0.005).

Muscle Mass Data

Muscle mass data for all 21 rabbits is shown in Table 1. The Botox injection used resulted in significant loss of muscle mass in the injected limbs. The only significant difference was that the rectus femoris muscle in the contralateral limb of the Botox+HMB group was significantly greater than the contralateral rectus femoris in the Botox alone group.

TABLE 1
All Rabbits Muscle Weights. (g)

| | | | | | | | Overall |
|---|---|---|---|---|---|---|---|
| | | | | | Botox + | | Treatment |
| | Control | SEM | Botox | SEM | HMB | SEM | P-Value |
| Vastus lateralis | 17.52 | 0.66 | 7.25 | 0.64 | 7.42 | 0.65 | 0.0001 |
| Injected | | | | | | | |
| Vastus lateralis | 17.97 | 0.59 | 17.46 | 0.57 | 18.35 | 0.58 | 0.56 |
| Contralateral | | | | | | | |
| Vastus medialis | 4.95 | 0.27 | 4.13 | 0.27 | 4.11 | 0.27 | 0.08 |
| Injected | | | | | | | |
| Vastus medialis | 5.14 | 0.21 | 5.21 | 0.21 | 5.18 | 0.21 | 0.97 |
| Contralateral | | | | | | | |
| Rectus femoris | 9.91 | 0.56 | 6.13 | 0.55 | 5.35 | 0.56 | 0.0001 |
| Injected | | | | | | | |
| Rectus femoris | 10.21 | 0.34 | 9.84 | 0.33 | 10.88^{a} | 0.34 | 0.11 |
| Contralateral | | | | | | | |
| Small vastus | 6.23 | 0.26 | 6.23 | 0.26 | 6.63 | 0.26 | 0.47 |
| lateralis | | | | | | | |
| Injected | | | | | | | |
| Small vastus | 5.68 | 0.35 | 6.14 | 0.34 | 6.38 | 0.34 | 0.39 |
| lateralis | | | | | | | |
| Contralateral | | | | | | | |
| Total weight | 38.61 | 1.14 | 23.75 | 1.11 | 23.51 | 1.13 | 0.0001 |
| Injected | | | | | | | |
| Total Weight | 39.00 | 1.05 | 38.65 | 1.02 | 40.78 | 1.03 | 0.32 |
| Contralateral | | | | | | | |

^{a}Significantly different from Botox p < 0.04. Least Square Means Analysis.

A subset of the 21 rabbits was chosen based upon the injected side musculature. The 2 rabbits having the lowest total weight musculature in each treatment group were dropped from this subset analysis due to the rather severe dosage of botox that was used. Therefore, muscle data from 15 of the 21 rabbits were analyzed. The muscle mass data from this subset appears in Table 2.

TABLE 2
Subset of Rabbits Muscle Weights. (g)

| | | | | | | | Overall |
|---|---|---|---|---|---|---|---|
| | | | | | Botox + | | Treatment |
| | Control | SEM | Botox | SEM | HMB | SEM | p-Value |
| Vastus lateralis | 18.39 | 0.65 | 7.85 | 0.63 | 7.82 | 0.62 | 0.0001 |
| Injected | | | | | | | |
| Vastus lateralis | 18.76 | 0.48 | 18.06 | 0.47 | 19.02 | 0.46 | 0.36 |
| Contralateral | | | | | | | |
| Vastus medialis | 5.12 | 0.35 | 4.08^{a} | 0.34 | 4.38^{b} | 0.33 | 0.15 |
| Injected | | | | | | | |
| Vastus medialis | 5.22 | 0.25 | 4.99 | 0.25 | 5.39 | 0.24 | 0.52 |
| Contralateral | | | | | | | |
| Rectus femoris | 9.95 | 0.64 | 6.94 | 0.63 | 5.79 | 0.61 | 0.002 |
| Injected | | | | | | | |
| Rectus femoris | 10.12 | 0.38 | 10.36 | 0.37 | 11.15^{c} | 0.36 | 0.16 |
| Contralateral | | | | | | | |
| Small vastus | 6.11 | 0.29 | 6.53 | 0.28 | 6.92^{d} | 0.28 | 0.18 |
| lateralis | | | | | | | |
| Injected | | | | | | | |
| Small vastus | 5.65 | 0.47 | 6.25 | 0.46 | 6.69 | 0.45 | 0.33 |
| lateralis | | | | | | | |
| Contralateral | | | | | | | |
| Total weight | 39.57 | 0.94 | 25.40 | 0.92 | 24.91 | 0.89 | 0.0001 |
| Injected | | | | | | | |
| Total Weight | 39.74 | 0.96 | 39.66 | 0.94 | 42.24^{e} | 0.91 | 0.12 |
| Contralateral | | | | | | | |

^{a}Tended to be different from Control p < 0.06. Least Square Means Analysis.
^{b}Not different from Control. Least Square Means Analysis.
^{c}Tended to be different from Control p < 0.08. Least Square Means Analysis.
^{d}Tended to be different from Control p < 0.07. Least Square Means Analysis.
^{e}Tended to be different from Control and Botox, p < 0.09 and p < 0.07, respectively. Least Square Means Analysis.

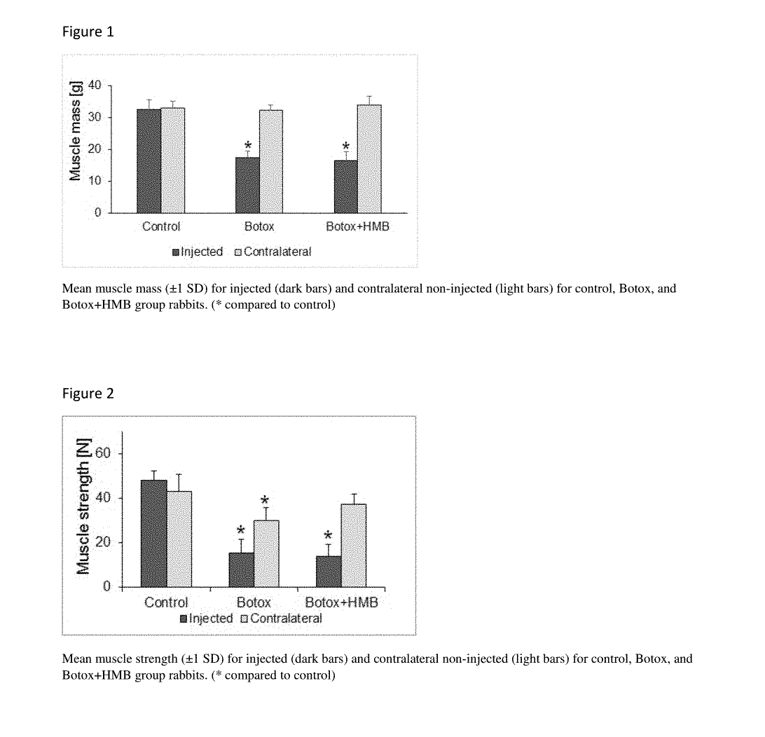

Injection with botox resulted in an approximately 36% decrease in muscle size for all muscles measured. Only the small vastus lateralis muscle was not affected by the botox injection. The injected leg vastus medialis muscle was larger in the botox+HMB group than in the botox alone group and a t-test of least square means differences showed that only the botox group was significantly different from the control group (p<0.05). In addition, the injected leg small vastus lateralis muscle in the botox+HMB group tended to be larger than in the control group (p<0.07). Least square means analysis of the botox+HMB group contralateral rectus femoris muscle tended to be greater than the rectus femoris muscle weights in the control group (p<0.08). The total weight of the measured muscles in the contralateral leg for the botox+HMB group tended to be greater than those in the botox alone group (p<0.07) and also tended to be greater than the total weight of the muscles in the control group (p<0.09). Additionally, the muscles affected the most by the botox injection (vastus lateralis, vastus medialis, and rectus femoris) weighed more in the contralateral leg for the botox+HMB group than the botox group (p<0.04). Expressing the muscle weights as a percentage of body weight showed the same effect, larger contralateral musculature with HMB versus the botox group (p<0.10) and the control group (p<0.03). These results are depicted in FIG. 1.

Supplementation with HMB helped to preserve muscle mass in the botox-injected rabbits. HMB greatly increased the muscle hypertrophy in the contralateral leg in the botox+HMB group when compared with both the botox and control groups. Thus, HMB did help preserve and hypertrophy muscles despite the severe nature of the botulinum toxin.

Muscle Strength Data

Isometric knee extensor strength was measured in the saline-injected (Control) or botox-injected and the contralateral musculature eight weeks following the botox or saline injections.

The femoral nerve was stimulated as described above and results of the stimulation at 100 and 200 Hz frequencies of stimulation appear in Tables 3 and 4.

TABLE 3
Mean Muscle force generated at 100 Hz stimulation. (N)

| | | | | | | | Overall |
|---|---|---|---|---|---|---|---|
| | | | | | Botox + | | Treatment |
| | Control | SEM | Botox | SEM | HMB | SEM | P-Value |
| 80 Degrees | 43.2 | 2.2 | 16.1 | 2.2 | 17.1 | 2.2 | 0.0001 |
| Injected | | | | | | | |
| 80 Degrees | 42.8 | 2.1 | 34.4^{a} | 2.0 | 39.2^{bc} | 2.1 | 0.03 |
| Contralateral | | | | | | | |
| 100 Degrees | 44.3 | 2.3 | 15.1 | 2.2 | 14.9 | 2.3 | 0.0001 |
| Injected | | | | | | | |
| 100 Degrees | 39.9 | 2.3 | 32.3^{a} | 2.3 | 35.2^{b} | 2.3 | 0.09 |
| Contralateral | | | | | | | |
| 120 Degrees | 34.7 | 2.2 | 11.9 | 2.1 | 13.1 | 2.2 | 0.0001 |
| Injected | | | | | | | |
| 120 Degrees | 33.2 | 2.0 | 29.7 | 1.9 | 30.0 | 2.0 | 0.42 |
| Contralateral | | | | | | | |

^{a}Significantly different from Control, p < 0.01 and p < 0.03 for 80 and 100 degrees respectively. Least Square Means Analysis.
^{b}Not significantly different from Controls. Least Square Means Analysis.
^{c}Tended to be different from Botox p < 0.11. Least Square Means.

TABLE 4
Mean Muscle force generated at 200 Hz stimulation. (N)

| | | | | | | | Overall |
|---|---|---|---|---|---|---|---|
| | | | | | Botox + | | Treatment |
| | Control | SEM | Botox | SEM | HMB | SEM | P-Value |
| 80 Degrees | 43.7 | 2.0 | 16.2 | 2.0 | 17.8 | 2.0 | 0.0001 |
| Injected | | | | | | | |
| 80 Degrees | 44.7 | 2.0 | 34.4 | 2.0 | 40.8^{ab} | 2.0 | 0.007 |
| Contralateral | | | | | | | |
| 100 Degrees | 47.6 | 2.4 | 15.7 | 2.4 | 16.0 | 2.4 | 0.0001 |
| Injected | | | | | | | |
| 100 Degrees | 43.1 | 2.0 | 30.2 | 1.9 | 37.3^{c} | 1.9 | 0.0008 |
| Contralateral | | | | | | | |
| 120 Degrees | 40.6 | 1.7 | 12.7 | 1.6 | 14.3 | 1.7 | 0.0001 |
| Injected | | | | | | | |
| 120 Degrees | 38.5 | 2.3 | 28.5 | 2.2 | 32.7^{b} | 2.3 | 0.02 |
| Contralateral | | | | | | | |

^{a}Significantly different from Botox p < 0.04. Least Square Means Analysis
^{b}Not significantly different from Controls. Least Square Means Analysis.
^{c}Significantly different from Botox p < 0.02. Least Square Means Analysis.

When compared with the control group, the overall decrease in strength for the botox injected muscles was similar for both the botox alone and botox+HMB groups. Both botox-injected groups lost between 63 and 66% of muscle strength as measured at both 100 Hz and 200 Hz stimulation frequencies. Contralateral muscle strength in the botox-injected rabbits decreased by 16.8 and 26.3% at 100 and 200 Hz, respectively, whereas contralateral strength in the botox+HMB rabbits decreased only about half as much by 9.9 and 12.3% at 100 and 200 Hz, respectively. At 100 Hz, HMB resulted in no statistically significant loss in strength in the contralateral muscles when compared with control rabbits, whereas there was a significant loss in strength in the contralateral muscles of the botox alone rabbits (p<0.01). At 200 Hz stimulation HMB treatment resulted in even greater preservation of strength in the contralateral muscles. Strength in the botox+HMB treated rabbits was significantly greater than in the botox alone group at 80 and 100 degrees, and at 80 and 120 degrees there was no significant difference in strength between the botox+HMB and control groups. In summary, botox injection resulted in a significant loss of strength not only in the injected musculature, but also in the contralateral musculature, and HMB prevented much of the botox-induced loss of strength in the contralateral musculature. These results are depicted in FIG. 2.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

1. A method of improving joint health, comprising administering to an animal in need thereof a composition comprising from about 0.01 to about 0.2 g of β-hydroxy-β-methylbutyric acid (HMB) per kilogram of body weight in a 24 hour period wherein administration of the composition to an animal in need thereof has at least one effect selected from the group comprising decreasing joint instability, increasing joint stability, decreasing joint stiffness, improving joint function, improving joint health, improving balanced movement and improving skeletal joint stability during muscle movement.

2. The method of claim 1, wherein said HMB is selected from the group consisting of its free acid form, its salt, its ester and its lactone.

3. The method of claim 2, wherein said salt is selected from the group consisting of a sodium salt, a potassium salt, a magnesium salt, a chromium salt, and a calcium salt.

4. A method of increasing strength, comprising administering to an animal having joint inflammation, joint damage, and/or joint injury a composition comprising from about 0.01 to about 0.2 g of β-hydroxy-β-methylbutyric acid (HMB) per kilogram of body weight in a 24 hour period, wherein administration of the composition to the animal in need thereof has the effect of increasing strength in the side of the body contralateral to the site of said joint inflammation, joint damage and/or joint injury.

5. The method of claim 4, wherein the effect of increasing strength in the animal's contralateral side results in improved balanced movement.

6. The method of claim 4, wherein said HMB is selected from the group consisting of its free acid form, its salt, its ester and its lactone.

7. A method of increasing muscle mass, comprising administering to an animal having joint inflammation, joint damage, and/or joint injury a composition comprising from about 0.01 to about 0.2 g of β-hydroxy-β-methylbutyric acid (HMB) per kilogram of body weight in a 24 hour period, wherein administration of the composition to the animal in need thereof has the effect of increasing muscle mass in the side of the body contralateral to the site of said joint inflammation, joint damage and/or joint injury.

8. The method of claim 7, wherein the effect of increasing strength in the animal's contralateral side results in improved balanced movement.

9. The method of claim 7, wherein said HMB is selected from the group consisting of its free acid form, its salt, its ester and its lactone.

10. A method of improving joint health, comprising administering to an animal in need thereof a composition comprising from about 0.5 g to about 30 g of β-hydroxy-β-methylbutyric acid (HMB) in a 24 hour period wherein administration of the composition to an animal in need thereof has at least one effect selected from the group comprising decreasing joint instability, increasing joint stability, decreasing joint stiffness, improving joint function, improving joint health, improving balanced movement and improving skeletal joint stability during muscle movement.

11. A method of increasing muscle mass, comprising administering to an animal having joint inflammation, joint damage, and/or joint injury a composition comprising from about 0.5 g to about 30 g of β-hydroxy-β-methylbutyric acid (HMB) in a 24 hour period, wherein administration of the composition to the animal in need thereof has the effect of increasing muscle mass in the side of the body contralateral to the site of said joint inflammation, joint damage and/or joint injury.

12. A method of increasing strength, comprising administering to an animal having joint inflammation, joint damage, and/or joint injury a composition comprising from about 0.5 g to about 30 g of β-hydroxy-β-methylbutyric acid (HMB) in a 24 hour period, wherein administration of the composition to the animal in need thereof has the effect of increasing strength in the side of the body contralateral to the site of said joint inflammation, joint damage and/or joint injury.
